(19) United States
(12) Patent Application Publication
(10) Pub. No.: US 2022/0371919 A1
(43) Pub. Date: Nov. 24, 2022

(54) DEVICE TO DISINFECT WATER USING ULTRAVIOLET LIGHT

(71) Applicant: Steribin, LLC, St. George, UT (US)

(72) Inventors: Wayne Provost, Ivins, UT (US); Jonathan M. Cole, St. George, UT (US); Jeffrey Mel Glauser, Santa Clara, UT (US)

(73) Assignee: Steribin, LLC, St. George, UT (US)

(21) Appl. No.: 17/745,656

(22) Filed: May 16, 2022

Related U.S. Application Data
(60) Provisional application No. 63/190,370, filed on May 19, 2021.

(51) Int. Cl. C02F 1/32

(52) U.S. Cl. CPC C02F 1/325; C02F 2303/04; C02F 2201/3228

(57) ABSTRACT

A water disinfecting device has a housing, an inlet, an outlet, a high-power disinfecting light, at least one quartz pipe that carries water, and a reflective surface for increased dosage. By placing multiple quartz pipes side by side, the device can disinfect greater quantities of water, or provide for double exposure of water passing through the pipes. By using a powerful UV light and a reflective surface from the outside of a quartz pipe, the device is capable of disinfecting at a much higher success rate than other disinfecting devices found in the prior art while also allowing for ease of maintenance.

### CROSS-REFERENCE TO RELATED APPLICATIONS

This application claims priority to U.S. Provisional Application Ser. No. 63/190,370, filed on May 19, 2021, which is incorporated herein by reference.

### TECHNICAL FIELD

The present disclosure relates to a device to disinfect water. More particularly, the present disclosure relates to a device to disinfect water and that provides a double-pass disinfection or single-pass disinfection.

### BACKGROUND

Infectious diseases commonly spread through the direct transfer of bacteria, viruses, or other microbes on surfaces or in water. Without disinfecting surfaces and water, many people may become ill from the numerous types of viruses and bacteria that surround us. To disinfect water and ensure it is safe to drink, people have turned to chemicals and filters. Some products use ultraviolet light to disinfect water. However, most of these UV devices use a low-power lamp that disinfects the water from inside of a quartz sleeve. As a result, the prior art devices are mostly used for smaller pipes. Accordingly, the prior art devices are not capable of having high-water flow. It is also difficult to replace the lamps within the quartz sleeve, which may result in broken quartz.

Accordingly, there is a need for a disinfectant device that is capable of disinfecting high volumes of water and that is easy to maintain. The present disclosure seeks to solve these and other problems.

### SUMMARY OF EXAMPLE EMBODIMENTS

In some embodiments, a water disinfecting device comprises a housing, an inlet, an outlet, a high-power disinfecting light, a quartz pipe that carries water, and a reflective surface for increased dosage. By placing multiple quartz pipes side by side, the device can disinfect greater quantities of water. In some embodiments, a plurality of water disinfecting devices may be arranged in sequence. For example, a first water disinfecting device may disinfect before entering a water filter so as to prevent bacteria growth in the filter. The water may then pass through a second water disinfecting device before it is supplied for use.

In some embodiments, the water disinfecting device can be installed in-line with a water supply. By using a powerful UV light (or other disinfecting light) and a reflective surface from the outside of a quartz pipe, the device is capable of disinfecting at a much higher success rate than other disinfecting devices found in the prior art. In some embodiments, the water disinfecting device comprises a plurality of pipes within the housing.

### BRIEF DESCRIPTION OF THE DRAWINGS

FIG. 1 illustrates a side, front perspective view of a device to disinfect water;

FIG. 2 illustrates side perspective longitudinal cross-sectional view of a device to disinfect water;

FIG. 3 illustrates a side, front perspective interior view of a device to disinfect water;

FIG. 4 illustrates a cross-sectional view of a device to disinfect water;

FIG. 5 illustrates a side, rear perspective interior view of a device to disinfect water;

FIG. 6 illustrates a side, front perspective view of a device to disinfect water; and

FIG. 7 illustrates a side, rear perspective view of a device to disinfect water.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.). While ultraviolet (UV) light is used as an example throughout, it will be appreciated that any light, or electromagnetic wavelength of light, capable of destroying or inhibiting the growth of microorganisms is contemplated herein as a “disinfecting light.”

As previously discussed, there is a need for a water disinfecting device that is capable of disinfecting high volumes of water and that is easy to maintain. The present water disinfecting device seeks to solve these and other problems.

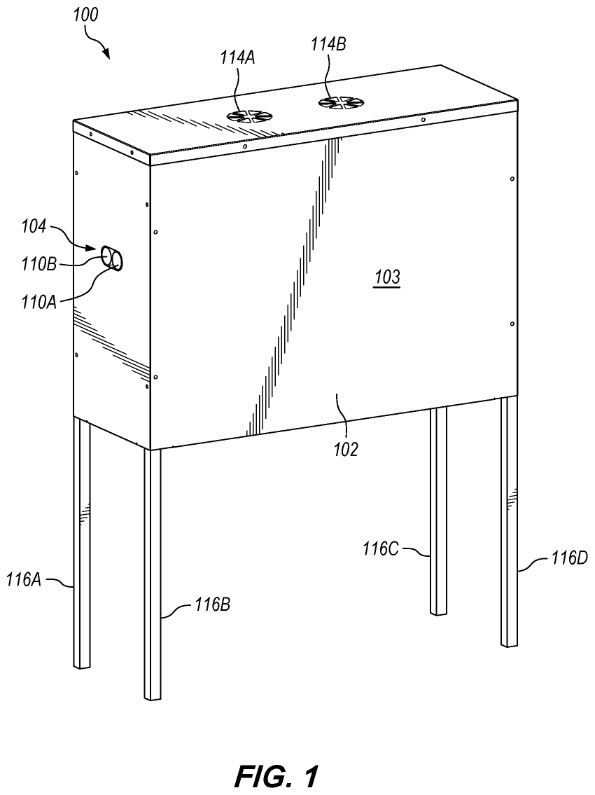

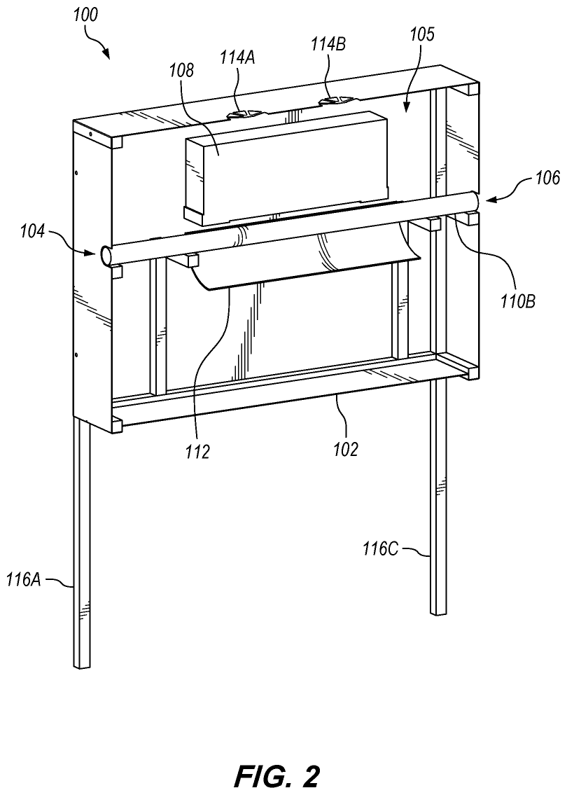

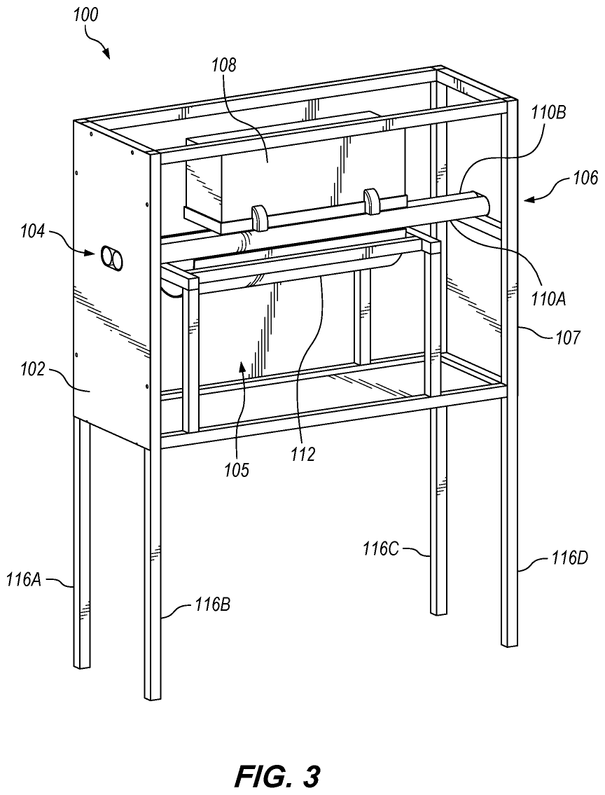

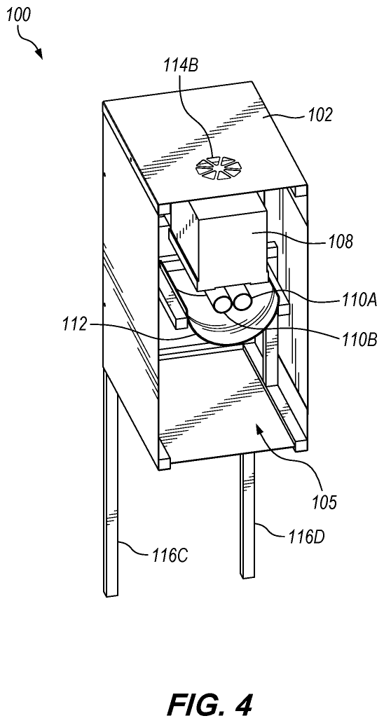

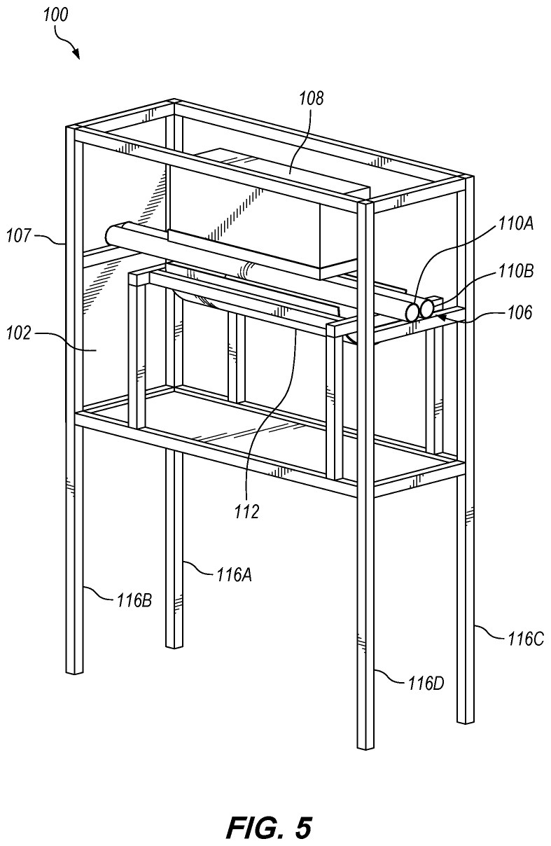

In some embodiments, as shown in FIGS. 1-5, a water disinfecting device 100 comprises a housing 102, an inlet 104, an outlet 106, a high-power pulse light or other disinfecting light 108, at least one quartz pipe 110A-B (best seen in FIGS. 3-5) that carries water, and reflective surface 112 for increased dosage (i.e., exposure to the disinfecting light 108. By placing multiple quartz pipes 110A-B in the housing 102, the device 100 can disinfect greater quantities of water in less time than devices in the prior art. For example, quartz pipes 110A-110B are positioned to run longitudinally through the housing 102 from the inlet 104 to the outlet 104. The disinfecting light 108 is positioned so as to expose the quartz pipe 110A-B with light. For example, as shown, the disinfecting light 108 may be mounted above the quartz pipes 110A-B. In this scenario, a reflective surface 112 may be placed on a bottom side of the quartz pipes 110A-B so that the quartz pipes 110A-B are interposed between the disinfecting light 108 and the reflective surface 112.

As shown, the reflective surface 112 may be configured in a half-pipe or arcuate shape so as to better direct the light from disinfecting light 112 to the underside of pipes 110A-B. The reflective surface 112 may be a mirror or mirror-like surface or comprise any suitable reflective material, such as, but not limited to, reflective aluminum, silver, or gold. As a result, as light passes downwardly from the disinfecting light 108, it is reflected upwardly toward the underside of pipes 110A-110B. This ensures that disinfecting light (e.g., UV light) surrounds the pipes 110A-B, thereby ensuring sufficient exposure so as to disinfect the water passing therethrough. Additionally, as will be discussed in more detail later herein, the same water may pass through a series of pipes, providing for multiple exposures to the disinfecting light 108.

In some embodiments, the disinfecting device 100 may comprise a plurality of disinfecting lights 108. For example, the reflective surface 112 may be replaced with a disinfecting light 108 so that the pipes 110A-B are interposed between the two disinfecting lights. It will be appreciated that the number and placement of disinfecting lights may vary without departing herefrom. Additionally, in some embodiments, the disinfecting device 100 may comprise a plurality of disinfecting lights 108 and one or more reflective surfaces 112. Further, the number and diameter of the pipes 110A-B may vary as well. In some embodiments with larger pipes or with a plurality of pipes, it may be preferred to have a plurality of disinfecting lights 108 so as to ensure adequate exposure.

In some embodiments, the water disinfecting device may comprise one or more circulation fans 114A-B for air circulation and to aid in heat dissipation from within the housing, thereby preventing overheating within the housing 102. Additionally, the housing 102 may be supported via one or more legs 116A-D.

In some embodiments, a plurality of water disinfecting devices 100 may be arranged in sequence. For example, a first water disinfecting device 100 may disinfect water before the water enters a water filter. This helps to prevent bacteria growth in the filter. The water may then pass from the filter through a second water disinfecting device 100 before it is supplied for use.

In some embodiments, the water disinfecting device 100 can be installed in-line with a water supply for commercial use, household use, or other use. By using a powerful UV light 108 and a reflective surface 112, both positioned externally to the quartz pipe 110A-B, the water disinfecting device 100 is capable of disinfecting at a much higher success rate than other disinfecting devices found in the prior art. This configuration also allows for maintenance and repairs on the disinfecting device with much greater ease and with less cost than the prior art. For example, a user may remove a panel 103 (FIG. 1) of housing 102 to access the interior 105 of the housing 102. In some embodiments, the panel 103 may be hingedly coupled to a frame 107, allowing for ease of access. With the panel 103 opened/removed, a user may easily repair or replace the disinfecting light 108 without needing to modify the pipes 110A-B or their connections. This reduces the risk of contamination within the pipes 110A-B as well as reduces the risk of breakage of the pipes 110A-B. Accordingly, the disinfecting device 100 may be easily and quickly serviced, allowing for minimal interruption to the flow of water.

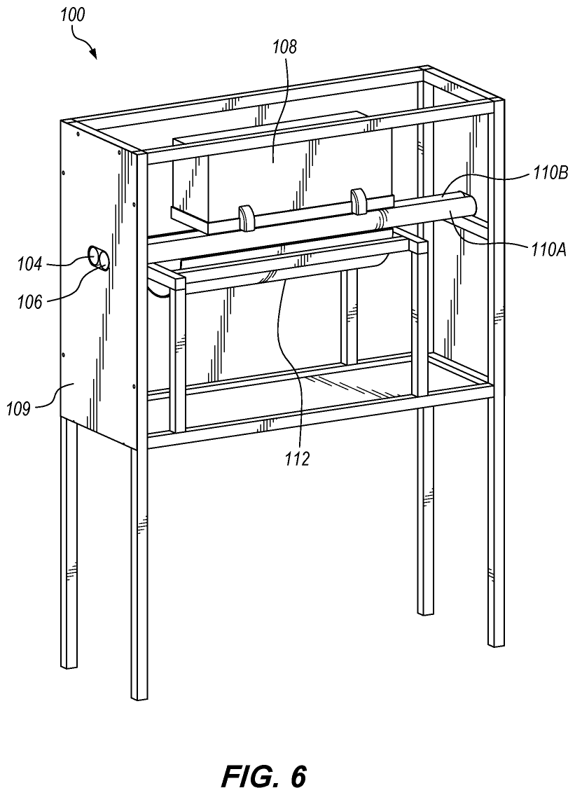

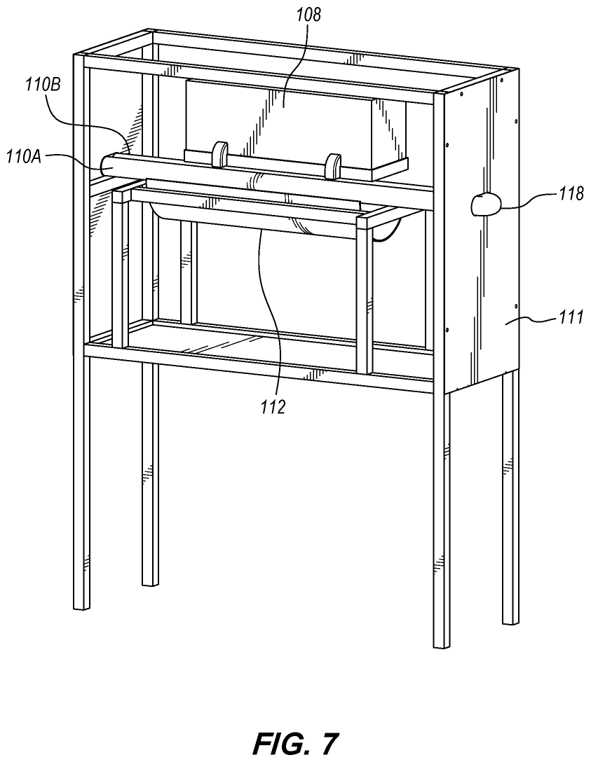

In some embodiments, the water may pass through the housing 102 twice for multiple exposures to the disinfecting light 108. For example, as shown in FIGS. 6-7, in some embodiments, a water disinfecting device 100 comprises an inlet 104 and an outlet 106 both on a first end 109 of the housing 102. For example, water enters the inlet 104 and flows through first pipe 110A where it is exposed to disinfecting light from at least one disinfecting light 108 and reflective surface 112. The water then passes through a pipe coupler 118 on a second end 111, which couples the first pipe 110A to the second pipe 110B, to then flow through second pipe 110B for a second exposure to the disinfecting light 108. This “double-pass” through the water disinfecting device 100 ensures that the water has been thoroughly disinfected without the need for a plurality of disinfecting devices 100, overcoming limitations of the prior art. While shown with two pipes 110A-B, it will be appreciated that the disinfecting device 100 disclosed herein may have as few as one pipe or two or more pipes, which may, or may not, be coupled together. Additionally, while shown as passing through pipe coupler 118 immediately upon exit of the housing 102, it will be appreciated that a filter may be interposed between the first pipe 110A and the second pipe 110B. In other words, water passes through first pipe 110A and is exposed to the disinfecting light 108, exits the housing 102, passes through a filter, and then is redirected back through the housing 102 via second pipe 110B, where it is exposed a second time to the disinfecting light 108. It will be appreciated that several configurations could be envisioned without departing herefrom.

Further, although generally referred to herein as a “disinfecting device,” it is understood that a disinfecting device of the present disclosure may disinfect, sterilize, sanitize, or otherwise treat and clean water or other liquids to achieve a lessened state or condition of contamination. Housing 102 may include an exterior material having a first property or function, and an interior material having a second property or function that is different than the first property or function. For example, in at least one embodiment, the housing 102 may include an exterior material that is structurally rigid and opaque, and an interior material that is reflective. In some embodiments, the interior material of the housing 102 may include a coating applied to an inner surface of the exterior material of the housing 102. Additionally, while generally described as a quartz pipe, it will be appreciated that the pipe may comprise glass, plastic, or other transparent materials.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A water disinfecting device, comprising:
a housing having an inlet and an outlet;
a quartz pipe extending in an interior of the housing from the inlet to the outlet;
at least one disinfecting light positioned in the interior of the housing on a first side of the pipe; and
at least one reflective surface positioned on a second, opposite side of the pipe and configured to reflect light from the at least one disinfecting light to the second side of the pipe.

2. The water disinfecting device of claim 1, further comprising at least one circulation fan.

3. The water disinfecting device of claim 1, further comprising a plurality of support legs.

4. The water disinfecting device of claim 1, wherein the at least one reflective surface comprises aluminum.

5. The water disinfecting device of claim 1, wherein the at least one reflective surface is arcuate.

6. A water disinfecting device, comprising:
a housing;
a first pipe in an interior of the housing, the first pipe extending from a first end of the housing to a second, opposite end of the housing;
a second pipe in the interior of the housing, the second pipe extending from a first end of the housing to a second, opposite end of the housing parallel to the first pipe;
a coupler configured to couple the first pipe to the second pipe at the second end of the housing;
at least one disinfecting light positioned in the interior of the housing on a first side of the first and second pipes; and
at least one reflective surface positioned on a second, opposite side of the first and second pipes and configured to reflect light from the at least one disinfecting light to the second side of the first and second pipes;
wherein water is configured to flow through the first pipe from the first end to the second end, through the coupler, and through the second pipe from the second end to the first end.

7. The water disinfecting device of claim 6, wherein the first and second pipes comprise quartz.

8. The water disinfecting device of claim 6, wherein the at least one reflective surface comprises aluminum.

9. The water disinfecting device of claim 6, wherein the at least one reflective surface is arcuate.

10. The water disinfecting device of claim 6, further comprising at least one circulation fan.

11. A method of disinfecting water using a water disinfecting device, the method comprising:
providing water to an inlet of the water disinfecting device, the water flowing through a quartz pipe within a housing of the water disinfecting device;
disinfecting the water as it passes through the quartz pipe by exposing the water to at least one disinfecting light, the disinfecting light within the housing and external to the quartz pipe; and
the water exiting the quartz pipe at an outlet.

12. The method of claim 11, wherein the water disinfecting device further comprises a reflective surface for reflecting light from the at least one disinfecting light for further exposing and thereby disinfecting the water as it flows through the quartz pipe.

13. The method of claim 11, wherein the water disinfecting device further comprises a second quartz pipe for disinfecting water therein using the at least one disinfecting light.

14. The method of claim 13, wherein the second quartz pipe is coupled to the first quartz pipe on a second end so that water flowing through the first quartz pipe subsequently flows through the second quartz pipe.
